(19) United States
(12) Patent Application Publication
(10) Pub. No.: US 2013/0103088 A1
(43) Pub. Date: Apr. 25, 2013

(54) Segmental Spinous Process Anchor System and Methods of Use

(71) Applicants: Dean Karahalios, Lake Forest, IL (US); Patrick Hunt, Denver, CO (US); Justin Taber, Lafayette, CO (US); Andrew Lamborne, Golden, CO (US); Greg Causey, Erie, CO (US)

(72) Inventors: Dean Karahalios, Lake Forest, IL (US); Patrick Hunt, Denver, CO (US); Justin Taber, Lafayette, CO (US); Andrew Lamborne, Golden, CO (US); Greg Causey, Erie, CO (US)

(73) Assignee: LANX, INC., Broomfield, CO (US)

(21) Appl. No.: 13/617,103

(22) Filed: Sep. 14, 2012

Related U.S. Application Data
(60) Provisional application No. 61/535,859, filed on Sep. 16, 2011.

(51) Int. Cl. A61B 17/70

(52) U.S. Cl. CPC A61B 17/7068
USPC 606/248; 606/279

(57) ABSTRACT

Segmental spinous process implant systems and methods of use are provided for coupling to one or more spinal processes of a cervical, thoracic, and/or lumbar spine. Embodiments of the segmental spinous process implant system include a support member coupled to one or more offset connector. The support member extends adjacent to vertebrae of a cervical, thoracic, and/or lumbar spine. The offset connector extends from the support member between adjacent spinous processes of the spine and supports a pair of spinous process connectors that secure the implant to a spinous process of a vertebra of the spine.

### PRIORITY

This application claims priority to U.S. Provisional Patent Application Ser. No. 61/535,859, filed on Sep. 16, 2011, titled Segmental Spinous Process Anchor System and Methods of Use, the disclosure of which is incorporated by reference as if set out in full.

### CROSS-REFERENCE TO RELATED APPLICATIONS

The present application is related to U.S. application Ser. No. 11/934,604, filed Nov. 2, 2007, entitled Spinous Process Implants and Associated Methods, the complete disclosure of which is incorporated herein by reference for all purposes.

### BACKGROUND

a. Field

The present invention relates to spinous process implants and associated methods.

b. Background

The vertebrae of the human spine are arranged in a column with one vertebra on top of the next. An intervertebral disc lies between adjacent vertebrae to transmit force between the adjacent vertebrae and provide a cushion between them. The discs allow the spine to flex and twist. With age, spinal discs begin to break down, or degenerate resulting in the loss of fluid in the discs and consequently resulting in them becoming less flexible. Likewise, the disks become thinner allowing the vertebrae to move closer together. Degeneration may also result in tears or cracks in the outer layer, or annulus, of the disc. The disc may begin to bulge outwardly. In more severe cases, the inner material of the disc, or nucleus, may actually extrude out of the disc. In addition to degenerative changes in the disc, the spine may undergo changes due to trauma from automobile accidents, falls, heavy lifting, and other activities. Furthermore, in a process known as spinal stenosis, the spinal canal narrows due to excessive bone growth, thickening of tissue in the canal (such as ligament), or both. In all of these conditions, the spaces through which the spinal cord and the spinal nerve roots pass may become narrowed leading to pressure on the nerve tissue which can cause pain, numbness, weakness, or even paralysis in various parts of the body. Finally, the facet joints between adjacent vertebrae may degenerate and cause localized and/or radiating pain. All of the above conditions are collectively referred to herein as spine disease.

Conventionally, surgeons treat spine disease by attempting to restore the normal spacing between adjacent vertebrae. This may be sufficient to relieve pressure from affected nerve tissue. However, it is often necessary to also surgically remove disc material, bone, or other tissues that impinge on the nerve tissue and/or to debride the facet joints. Often, the restoration of vertebral spacing is accomplished by inserting a rigid spacer made of bone, metal, or plastic into the disc space between the adjacent vertebrae and allowing the vertebrae to grow together, or fuse, into a single piece of bone. The vertebrae are typically stabilized during this fusion process with the use of bone plates and/or pedicle screws fastened to the adjacent vertebrae.

Although techniques for placing intervertebral spacers, plates, and pedicle screw fixation systems have become less invasive in recent years, they still require the placement of hardware deep within the surgical site adjacent to the spine. Recovery from such surgery can require several days of hospitalization and long, slow rehabilitation to normal activity levels.

More recently, another such implant is the spinous process spacer which is inserted between the posteriorly extending spinous processes of adjacent vertebrae to act as an extension stop and to maintain a minimum spacing between the spinous processes when the spine is in extension. The spinous process spacer allows the adjacent spinous processes to move apart as the spine is flexed.

In some cases, a patient may need additional surgery on a level adjacent to vertebrae that have been previously fused. In some cases, the patient may receive additional pedicle screws in the adjacent level, and a longer longitudinal rod to span the levels of both surgeries.

### BRIEF SUMMARY

In some embodiments, a spinous process implant is provided. The implant includes a support member having a longitudinal axis, and an offset connector coupled to the support member. The offset connector includes an anchor, for selectively coupling the offset connector along the support member, and an offset member having a longitudinal axis extending at an angle away from the longitudinal axis of the support member. The offset member is operable to extend laterally across a spine adjacent to at least one spinous process. The implant includes a pair of opposing spinous process connectors operable to engage the spinous process. The spinous process connectors are coupled to the offset member and extend away from the offset member to be generally alongside either side of the spinous process. At least one of the spinous process connectors is movably coupled to the offset member so as to be movable with respect to the other opposing spinous process connector to secure the spinous process between the pair of opposing spinous process connectors.

In another embodiment, a bilateral spinous process implant is provided. The implant includes a first support member having a first longitudinal axis and a second support member having a second longitudinal axis, with the second support member spaced apart from the first support member. The implant includes an offset connector having (i) a first anchor for selectively coupling the offset connector to the first support member along the first longitudinal axis, (ii) a second anchor for selectively coupling the offset connector to the second support member along the second longitudinal axis, and (iii) an offset member having a longitudinal axis extending between the first and second support members. The offset member is operable to extend laterally across a spine adjacent to at least one spinous process. The implant further includes a pair of opposing spinous process connectors operable to engage the spinous process. The pair of opposing spinous process connectors is coupled to the offset member and extend away from the offset member to extend generally alongside either side of the spinous process. At least one of the pair of opposing spinous process connectors is movably coupled to the offset member so as to be movable with respect to the other opposing spinous process connector to secure the spinous process between the pair of opposing spinous process connectors.

Methods of using a spinous process implant are provided. One such method includes providing an implant having a first elongate support member, an offset connector and a pair of spinous process connectors. The method includes slidably engaging the first elongate support member with the offset connector so that the offset connector is generally transverse to the elongate support member, and slidably engaging the pair of spinous process connectors with the offset connector, with the pair of spinous process connectors extending generally transverse to the offset connector. The method includes engaging a spinous process with the pair of spinous process connectors and fixing the position of the spinous process connectors to the offset connector to maintain the engagement with the spinous process. The method includes fixing the position of the offset connect to the first elongate support member.

The foregoing and other aspects, features, details, utilities, and advantages of the present invention will be apparent from reading the following description and claims, and from reviewing the accompanying drawings.

### BRIEF DESCRIPTION OF THE DRAWINGS

Various examples of a modular spinous process implant will be discussed with reference to the appended drawings. These drawings depict only illustrative examples of the invention and are not considered to be limited in scope.

FIG. 1 is a side partial cross-sectional view of an example modular spinal process implant in situ.

FIG. 2 is a side elevational view of the implant of FIG. 1 in situ.

FIG. 3 is front elevational view of the implant of FIG. 1.

FIG. 4 is an exploded perspective view of the implant of FIG. 1.

FIG. 5 is an exploded perspective view of an example offset connector the implant of FIG. 1.

FIG. 6 is an exploded perspective view of an example spinous process connector comprising pair of spinous process spiked plates of the implant of FIG. 1.

FIG. 7 is a front elevational view of another example modular spinal process implant.

FIG. 8 is an exploded perspective view of the implant of FIG. 7.

FIG. 9 is a perspective view of an open anchor of the implant of FIG. 7.

### DETAILED DESCRIPTION

A segmental spinous process implant system is provided for coupling one or more spinal processes of a cervical, thoracic, and/or lumbar spine. Embodiments of the segmental spinous process implant system include a support member coupled to one or more offset connectors. The support member extends adjacent to one or more vertebrae of a cervical, thoracic, and/or lumbar spine. The offset connector extends from the support member between adjacent spinous processes of the spine and supports a pair of spinous process connectors that secure the implant to one or more spinous processes of the spine.

The support member, offset connector, and spinous process connectors may be provided in a variety of sizes to accommodate anatomical variation amongst patients and varying degrees of space correction. The offset connectors may be coupled anywhere along the support member to provide variable longitudinal spacing between offset connectors to accommodate anatomical variation amongst patients, and/or variation in the desired spacing between vertebra.

In some embodiments, at least one of the pair of spinous process connectors is movable with respect to the other spinous process connector to secure the spinous process between the pair of spinous process connectors. In one embodiment, for example, both of the spinous process connectors can slide along an offset member (e.g., an offset rod or other shaped offset member) of the offset connector to move with respect to the other spinous process connector and to secure the spinous process between the pair of spinous process connectors. In this embodiment, the spinous process connectors can provide variable lateral spacing for connecting to spinous processes of the spine that may not be aligned. In some embodiments, spinous process connectors are coupled to a spinous process, and the spinous process connector then may be moved to compress or distract the spinous process relative to an adjacent spinous process.

In some embodiments cerclage may be used to stabilize the spinous process implant and/or to provide other benefits. For example, wires, straps, bands, cables, cords, and/or other elongated members may encircle the pedicles, laminae, spinous processes, transverse processes, and/or other spinal structures. The cerclage may be relatively inextensible to provide a hard check to spine flexion or the cerclage may be relatively extensible to provide increasing resistance to flexion. The cerclage may be relatively flexible and drapeable such as a woven fabric or it may be relatively rigid such as a metal band. The cerclage may have shape memory properties that cause it to resume a prior set shape after implantation. The cerclage may be independent of the spinous process implant or may engage it. For example, the cerclage may pass through a hollow interior of the spinous process implant and/or engage the extension. The cerclage may be offset from the spacer and provide a tensioning force that uses the spacer as a fulcrum to offload the disc and/or open the disc space. Additional details on cerclage for use with the present embodiments are disclosed in U.S. application Ser. No. 11/934,604, previously incorporated herein by reference.

In some embodiments, a bone graft or a bone growth promoting substance is placed in the interspinous space and/or surrounding the implant to help facilitate bony growth or fusion. The implant and any associated cerclage or other components may be made of any suitable biocompatible material including among others metals, resorbable ceramics, non-resorbable ceramics, resorbable polymers, and non-resorbable polymers. Some specific examples include stainless steel, titanium and its alloys including nickel-titanium alloys, cobalt chrome alloy, tantalum, hydroxylapatite, calcium phosphate, bone, zirconia, alumina, carbon, bioglass, polyesters, polylactic acid, polyglycolic acid, polyolefins, polyamides, polyimides, polyacrylates, polyketones, fluropolymers, and/or other suitable biocompatible materials and combinations thereof.

The spinous process implant may be used to treat spine disease in a variety of surgical techniques including superspinous ligament sacrificing posterior approaches, superspinous ligament preserving posterior approaches, lateral approaches, and/or other suitable approaches. The spinous process implant may be used to treat spine disease by fusing adjacent vertebrae or by preserving motion between adjacent vertebrae. It may include only an extension stop such as a spacer, only a flexion stop such as flexible cerclage elements, or both a flexion and extension stop. The spinous process implant may be used to reduce loads on the facet joints, increase spinous process spacing, reduce loads on the disc, increase anterior disc spacing, and/or otherwise treat spine disease. Anterior effects may be accomplished by tensioning spine elements posterior to the spacer to apply a mechanical advantage to the spinal construct. Techniques for the spinal process implant may include leaving the tissues at the surgical site unmodified or modifying tissues such as trimming, rasping, roughening, and/or otherwise modifying tissues at the implant site.

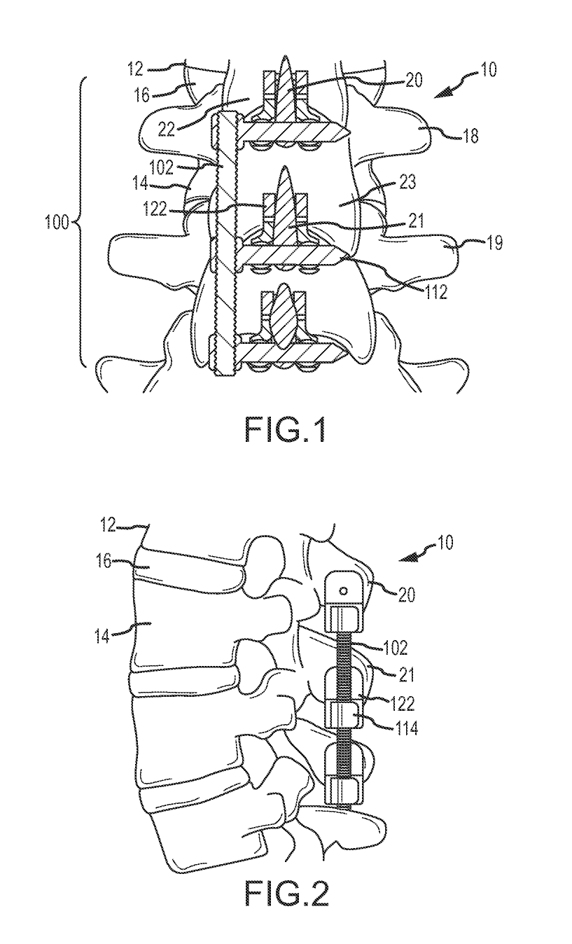

FIGS. 1 and 2 depict posterior and lateral views of a pair of adjacent vertebrae of a lumbar spine 10. A superior vertebra 12 is separated from an inferior vertebra 14 by a disc 16. Each vertebra includes a pair of transverse processes 18, 19, a posteriorly projecting spinous process 20, 21, and a pair of laminae 22, 23 connecting the transverse processes 18, 19 to the spinous process 20, 21. In addition to the connection through the disc 16, the vertebrae 12, 14 articulate at a pair of facet joints 24.

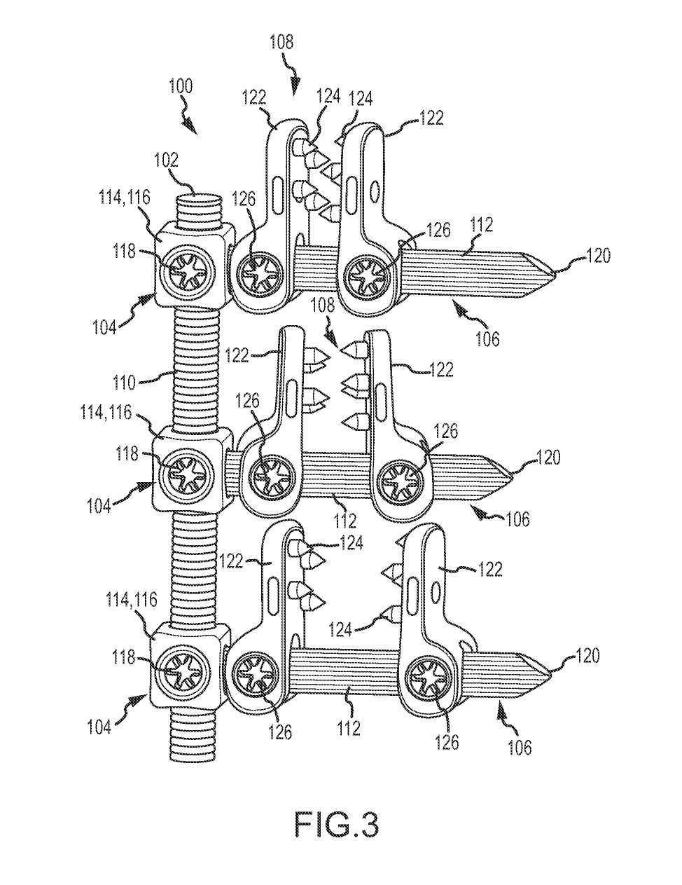

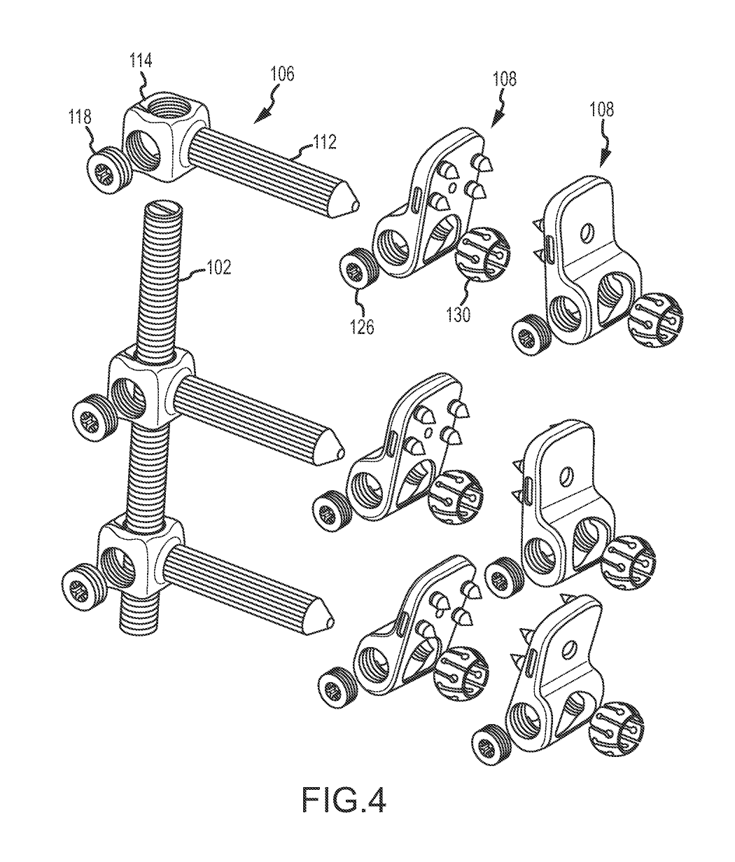

FIGS. 1-6 illustrate an example embodiment of a segmental spinous process implant 100. In the embodiment shown in FIGS. 1-6, the implant 100 includes a support member 102 providing one or more adjustable connection locations 104 for coupling to an offset connector 106. The offset connector 106, in turn, supports a pair of spinous process connectors 108 for coupling to posteriorly projecting spinous process 20, 21, such as shown in FIGS. 1 and 2.

The support member 102, for example, may comprise a generally longitudinal support rod or other shaped support member that may be surgically inserted generally alongside one or more spinous process 20, 21. In one embodiment, for example, the support member 102 may be bendable or flexible to conform to a shape of the spine. In the embodiment shown in FIGS. 1-6, the support member 102 is shown having a knurled surface 110 for connection to the offset connector 106. The knurled surface 110 of the support member 102, for example, may comprise a ring-shaped knurling as shown in FIGS. 1-6. In other embodiments, however, the surface of the support member 102 may comprise other knurling configurations, such as but not limited to, a diamond-shaped (criss-cross) pattern, helix shaped pattern or any other configuration. The support member 102 may alternatively comprise a smooth or textured surface to which an offset connector 106 may be coupled. In other embodiments, a second material is coated to the support member 102, the connector 106, or other system components to aid in the interaction therebetween. In a particular embodiment, support member 102 and/or connector 106 include a titanium plasma spray coating. In this manner, the components have an increased frictional resistance between them. The support member 102 may comprise any cross-sectioned shape. In one embodiment, the support member 102 comprises a round 5.5 mm rod, such as a titanium alloy (e.g., a TI-6AL-4V ELI titanium alloy) or cobalt chrome alloy rod. In alternative embodiments, support member 102 may have a different diameter, be made from a different material and have a variety of lengths. The support member 102, however, may also have a cross-section adapted to assist in locking an offset connector 106 to the support member 102. In one embodiment, for example, the support member 102 may comprise a flat surface on which a set screw may be tightened. In an alternative embodiment, support member 102 comprises PEEK, PAEK, or other similar material. In this manner, support member 102 may provide some dynamic stabilization characteristics at the vertebral segments to which support member 102 is coupled.

In the embodiment shown in FIGS. 1-6, the offset connector 106 comprises an offset rod 112 and an anchor 114 for coupling to the support member 102. The anchor 114, for example, may comprise a slide anchor 116 (e.g., the closed slide anchor shown in FIGS. 1-6) configured to slide along the support member 102 and be fixed to the support member 102 at a desired location along the support member 102. In other embodiments, the anchor 114 may comprise an open anchor (e.g., a hook anchor, a U-shaped anchor, etc.) that can be coupled to the support member 102 and fixed to the support member at a desired location along the support member 102.

The offset rod 112 of the offset connector 106 can be integral with or connected to the anchor 114. For example, offset rod 112 may be integrally formed with anchor 114 such that coupling anchor 114 to support member 102 operates to couple offset rod 112 to support member 102. In another embodiment, for example, the offset rod 112 can extend into an opening of the anchor 114 and be fixed to the anchor 114 via a set screw or other connector. Although the offset rod 112 is shown in FIGS. 1-6 as being coupled generally transverse to the support member 102, the offset rod 112 may be disposed in any other configuration to extend laterally across the spine or between spinous processes of the spine. In addition, although the offset rod 112 is shown as a straight rod in FIGS. 1-6, the rod may be bendable, flexible or variously shaped to conform to various anatomical features of different spines. In the illustrated embodiment, for example, the offset rod 112 comprises a tapered tip 120 to assist in guiding the offset rod between spinous processes of the spine during implantation.

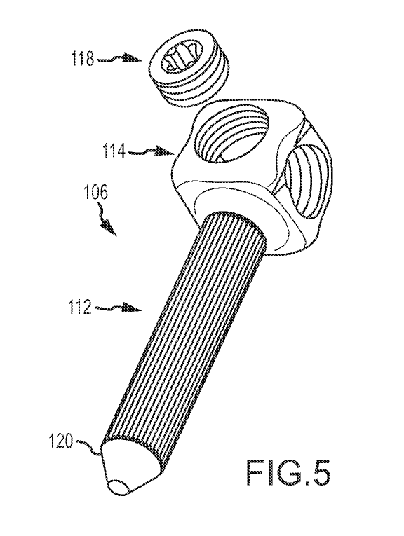

In the embodiment shown in FIGS. 1-6, the anchor 114 is fixed into place on the support member 102 by tightening a set screw 118 against the support member 102. FIG. 5 depicts an exploded perspective view of an example offset connector 106 of implant 100. As described above, the support member 102 may include knurling 110 or a textured surface. In these embodiments, an end of the set screw 118 may comprise a mating structure (e.g., teeth, protrusions, or the like) adapted to mate with knurling on the support member 102 or otherwise enhance the fixation of the anchor 114 to the support member 102. In the embodiment shown in FIG. 5, for example, a wavy pattern disposed on a distal end of the set screw 118 secures the tip of the set screw 118 to a ring knurling pattern 110 on the support member 102. In some embodiments, the wavy profile of set screw 118 is similar to the knurled or ringed profile of support member 102, with the waves extending radially from the surface of set screw 118. In this manner, the pattern of screw 118 helps to secure screw 118 to support member 102.

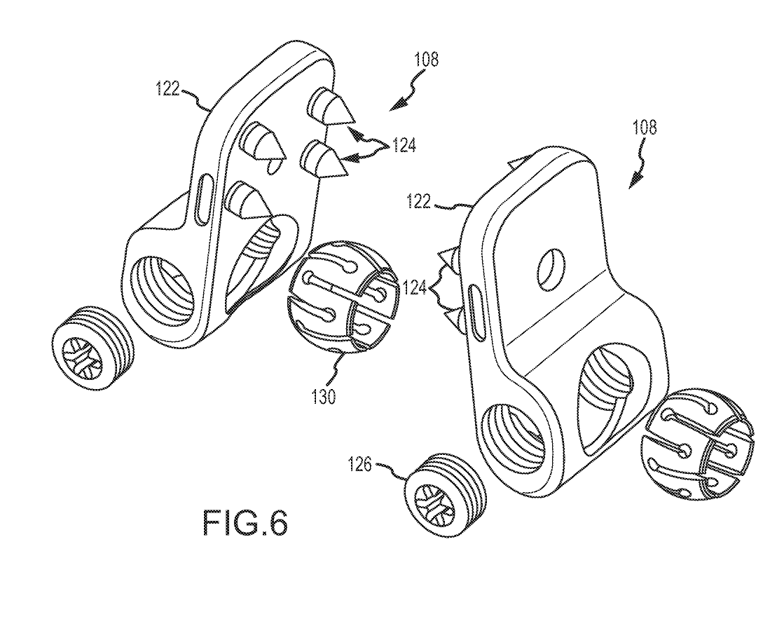

FIG. 6 depicts an exploded perspective view of an example spinous process connector 108 comprising a pair of spinous process spiked plates 122 of the implant 100. A pair of spinous process connectors 108 is coupled to the offset rod 112 of the offset connector 106. At least one of the pair of spinous process connectors 108 is slidably coupled to offset rod 112 and adapted to move axially along offset rod 112 to secure the spinous process, such as a superior or inferior spinous process, between the pair of spinous process connectors 108. In the embodiment shown in FIGS. 1-6, the spinous process connectors 108 each comprise a spinous process spiked plate 122 oriented to generally face each other. In this embodiment, each of the spinous process spiked plates 122 is movable axially with respect to each other along the offset rod 112 to secure the spinous process between the pair of spinous process spiked plates 122. In the depicted embodiment, each spinous process spiked plate 122 comprises fasteners 124 projecting from the spinous process spiked plate 122 toward the other spinous process spiked plate 122. While plates 122 are referred to herein as spiked plates 122, in alternative embodiments, only one of the pair of plates 122 may comprise fasteners 124. The fasteners 124 engage the spinous process to fix the spinous process between the pair of spinous process spiked plates 122. The spinous process connector 108 is fixed or coupled to the offset connector 106 by tightening a set screw 126 or other locking member. As discussed above with respect to the support member 102, the offset rod 112 of the offset connector 106 may include textured (e.g., knurled) or smooth surface 128 for connection to the spinous process connectors 108. Similarly, the surface of the offset rod 112 may comprise any cross-section shape to assist in locking a spinous process connector 108 to the offset rod 112. In one embodiment, for example, the offset rod 112 may comprise a flat surface on which a set screw may be tightened.

The fasteners 124 may include sutures, wires, pins, straps, clamps, spikes, screws, teeth, adhesives, roughened surfaces of plate 122, and/or other suitable fasteners. The fasteners 124 may be integrated into the plates 122 or they may be modular. Fasteners 124 may be the same for each plate 122 in a pair of plates 124, or they may differ between plates 122 in the pair. Modular fasteners may be adjustable, replaceable, and/or removable to allow tailoring of the kind and quality of fixation from rigid fixation to no fixation. The spinous process spiked plate 122 and fasteners 124 may advantageously be made of different materials. For example, the spinous process spiked plate 122 may be made of a relatively softer material while the fasteners 124 may be made of a relative harder material. For example, the spinous process spiked plate may be made of a polymer and/or other relatively soft material and the fastener may be made of a metal and/or other relatively hard material.

The fasteners 124 may take any suitable form. They may be made integral with the spinous process spiked plates 122, such as by machining or casting them with the plates 122, or they may be formed separately and permanently or removably attached to the spinous process spiked plates 122. In one embodiment, for example, fastener 124 is a sharpened spike that threadably engages the plate 122. The threaded engagement allows the fastener 124 to be replaced with a different fastener. For example, the fastener 124 may be replaced by one that has a different shape, a different size, a different material, or a different surface coating. The threaded engagement also allows the fastener 124 to be adjusted to extend by varying amounts from the plate 122 to vary how it engages the bone. Thus, the fastener 124 can be adjusted to fit differently shaped bones or to penetrate into a bone by varying amounts. For example, multiple threaded fasteners 124 can be adjusted to extend by different amounts to conform to curved or angled bone. Finally, the threaded engagement allows the user to remove the fastener 124 when fixation is not desired such as when it is desired to use implant 100 in a non-fusion procedure as an extension stop without limiting flexion. In another embodiment, implant 100 is configured for a dynamic application. In this case, plates 122 may have generally flat surfaces without spikes to engage the spinous process. A motion preserving band or cerclage may be used to couple plates 122 to the spinous process while still allowing at least some motion between adjacent spinous processes. Alternatively or additionally, a dynamic rod may be used to allow for some motion preservation at the vertebral segment. In a particular embodiment, support member 102 comprises PEEK or other similar materials.

Fasteners 124 can also be provided as multi-spike pods allowing a plurality of spikes to be quickly adjusted, changed, or omitted. Fastener 124 may include a non-circular tab engageable with a non-circular opening in the plate 122. The non-circular engagement prevents the fastener 124 from rotating. The tab may form a press-fit, snap-fit, or other suitable engagement with the opening. The tab may be further secured by a supplemental screw. In some embodiments fastener 124 includes a threaded shaft threadably engaged with a base member to allow the length of the fastener to be adjusted. The shaft engages the plate 122 in rotating and pivoting manner such that the fastener 124 can be adjusted rotationally and angularly to engage the bone surface. In one embodiment, the shaft terminates in a spherical ball that engages the opening in a ball-and-socket arrangement for three degrees of freedom. However, any mechanism that allows any number of degrees of freedom may be used. The fastener 124 may be allowed to move in use so that as the plate 122 is pressed toward a bone the fastener 124 adjusts to the angle of the bone surface. The fastener 124 may also be secured such as by screw to adjust the tension in the joint and/or to lock the fastener 124 in a predetermined orientation.

In alternative embodiments, fasteners 124 and plates 122 may have different arrangements. For example, in one embodiment plates 122 are adapted to ratchet along offset rod 112 to provide a single step locking function. In this manner, one or both plates 122 can be moved towards the spinous process and the ratcheting relationship between plates 122 and offset rod 112 operate to maintain the plates 122 in the adjusted position relative to the spinous process. Alternatively or additionally, plates 122 may be adjusted through a scissors-like alligator clip, by crimping relative to offset rod 112, or the like.

In one embodiment, the pair of spinous process connectors 108 is coupled to the offset connector 106 via a ball socket 130 allowing freedom of movement to angle and/or rotate the spinous process spiked plates 122 with respect to the offset connector 106. The freedom of movement provided by the ball socket connection between the spinous process connectors 108 and the offset connector 106 allow the spinous process spiked plates 122 to be positioned to conform to curved or angled bone of the spinous process. In one embodiment, for example, the spinous process spiked plates 122 are able to be angled at least about ±20 degrees with respect to the offset connector 106. Such an arrangement provides for a polyaxial cone of angulation of plate 122 about offset connector 106. Other connections allowing similar, more, or less, freedom of movement for the spinous process spiked plates 122 to be angled and/or rotated with respect to the offset connector 106 could also be provided. For example, the joint in the connection between the offset connector 106 and the spinous process spiked plates 122 may include enough free space through which the spinous process spiked plates may be angled and/or rotated with respect to the offset connector 106.

The segmental spinous process implant 100 provides a flexible implant system that may be implanted in a patient in many configurations. The ability to longitudinally adjust the offset connector 106 along the support member 102 provides the ability to compress or distract disc space. For example, the spiked plates 122 may be coupled or seated to the spinous process, such as by compressing fasteners 124 into the spinous process cortical bone. The spiked plates 122 may be coupled to the offset connector 106, such as with set screw 126. If desired, lateral movement of spinous process connectors 108 may occur to provide lateral forces to or movement of the spinous process. The compression or distraction of two adjacent spinous processes then may occur by adjusting the position of offset connector 106 along support member 102. In this manner, the distance between adjacent spinous processes may be adjusted, and then maintained.

In addition, the spinous process implant 100 provides for multilevel constructs with a single rigid construction to connect and secure multiple spinous processes. The spinous process implant 100 further provides segmental spinal process anchors with connectors that allow fixation of a spinous process to one or more other spinous processes. Each spinal process anchor allows for independent fixation and manipulation of spinous processes (e.g., compression or distraction) and independently adjustment of the spinous process connectors at spinous processes of different vertebrae.

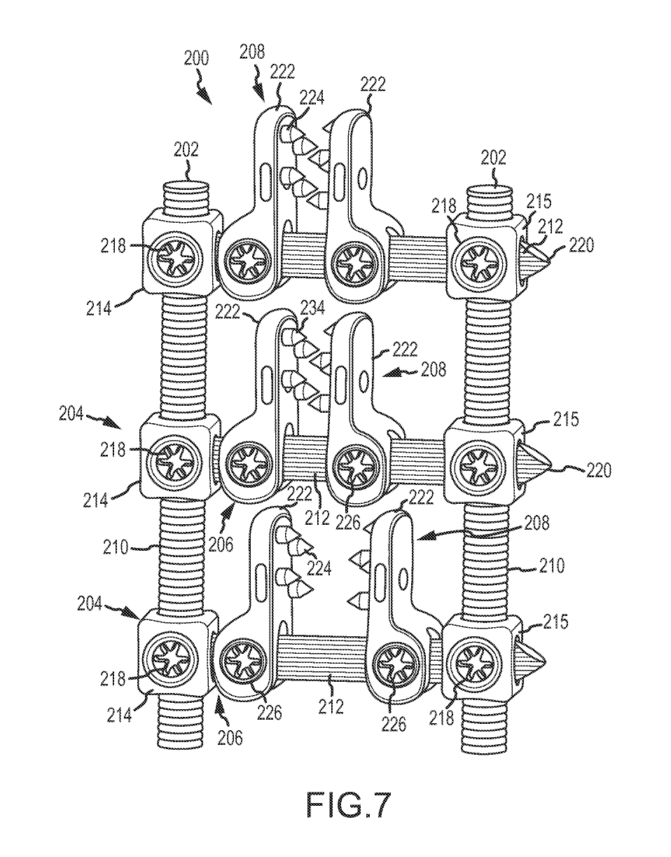

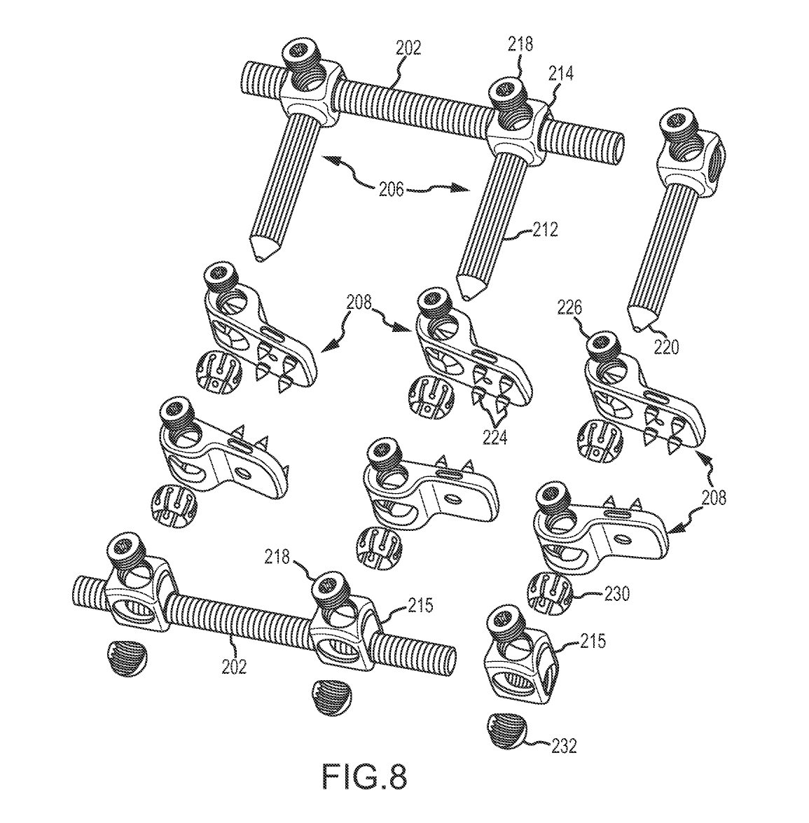

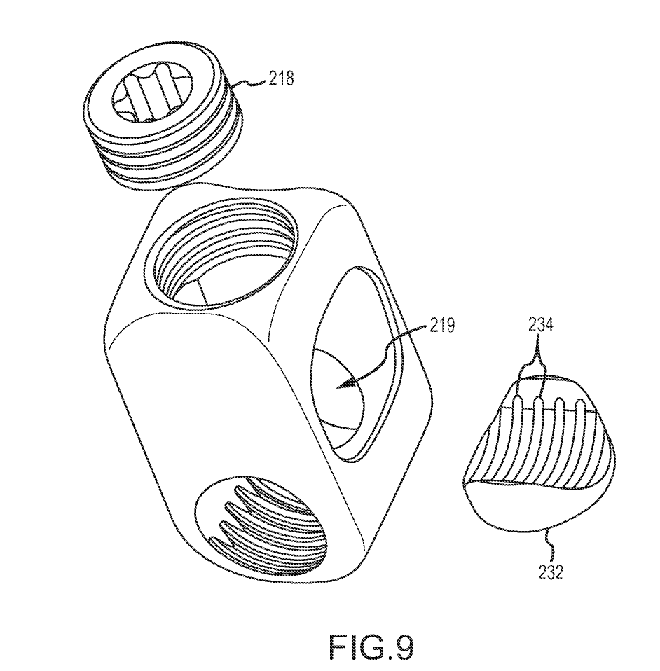

FIGS. 7-9 depict another example embodiment of a segmental spinous process implant 200 comprising bilateral support members 202. In this embodiment, bilateral support members 202 of the implant 200 comprise a pair of generally parallel support members 202 coupled to a plurality of offset connectors 206 at a plurality of adjustable connection locations 204 disposed along the length of the support member 202. Each offset connector 206, in turn, supports a pair of spinous process connectors 208 for coupling to a posteriorly projecting spinous process 20, 21, such as shown in FIGS. 1 and 2.

In some embodiments, the segmental spinous process implants 200 are similar in features and functionality as the segmental spinous process implants 100 discussed in conjunction with FIGS. 1-6. At least some of the description of the various components of implants 100 are applicable to the like components of implants 200.

In the embodiment shown in FIGS. 7-9, the support members 202, for example, may comprise a generally longitudinal support rod or other shaped support member that may be surgically inserted generally alongside one or more spinous process. Although the support members 202 are shown as generally straight and described as generally parallel, the individual support members 202 may be bent or otherwise altered in shape to conform to accommodate anatomical variation amongst patients. In this embodiment, the use of two support members 202 may provide additional stability to offset connectors 206, and thus to spinous process connectors 208. In the embodiment shown in FIGS. 7-9, the support members 202 are shown having a knurled surface 210 for connection to the offset connectors 206. As described above with respect to FIGS. 1-6, the knurled surface 210 of the support member 202 may comprise any number of patterns or textures (e.g. a ring-shaped knurling as shown in FIGS. 7-9, a diamond-shaped (criss-cross) pattern, helix shaped pattern, smooth surface, or any other configuration). The support member 202 may comprise any cross-sectioned shape. In one embodiment, the support member 202 comprises a round 5.5 mm rod, such as a titanium alloy (e.g., a TI-6AL-4V ELI titanium alloy) or cobalt chrome alloy rod. Support members 202 may further comprise PEEK rods, or rods comprised of other biocompatible plastics. The support member 202, however, may also have a cross-section adapted to assist in locking an offset connector 206 to the support member 202. In one embodiment, for example, the support member 202 may comprise a flat surface on which a set screw may be tightened.

In the embodiment shown in FIGS. 7-9, the offset connector 206 comprises an offset rod 212 and a pair of anchors 214, 215 for coupling to the support members 202. The anchors 214, 215, for example, may comprise a slide anchor configured to slide along the support member 202 and be fixed to the support member 202 at a desired location along the support member 202. In the embodiment shown in FIGS. 7-9 the anchors comprise a closed slide anchor 214 disposed on a first side of the implant 200 and an open slide anchor 215 disposed on a second side of the implant 200 as shown in FIGS. 7-9. The open slide anchor 215 comprises an opening 219 through which a tip 220 of the offset rod 212 is extended into and fixed within the open slide anchor 215 via a fastener such as a set screw 218. In some embodiments, anchor 215 includes a seat portion 232 adapted to rest within anchor and engage offset rod 212. Seat portion 232 may include one or more slots or ridges 234 which help engage offset rod 212. For example, as depicted, seat portion 232 has a plurality of curved slots which are adapted to mate with a textured or slotted surface of offset rod 212. In this manner, the tightening of set screw 218 helps to couple offset rod 212 within anchor 215 by having offset rod 212 engage the slots 234 within seat portion 232. In other embodiments, the anchors 214, 215 may comprise an open anchor (e.g., a hook anchor) that can be coupled to the support member 202 and fixed to the support member at a desired location along the support member 202.

The offset rods 212 of the offset connector 206 can be integral with or connected to one or more of the anchors 214, 215. In one embodiment, for example, the offset rods 212 can extend into an opening of the closed anchor 214 and be fixed to the closed anchor 214 via a set screw or other connector. Although the offset rods 212 are shown in FIGS. 7-9 as being coupled generally transverse to the pair of support members 202, the offset rods 212 may be disposed in any other configuration to extend between spinous processes of the spine. In addition, although the offset rods 212 are shown as a straight rod in FIGS. 7-9, the rods may be bendable, flexible or variously shaped to conform to various anatomical features of different spines. In the illustrated embodiment, for example, the offset rods 212 comprise a tapered tip 220 to assist in guiding the offset rods 212 between spinous processes of the spine during implantation.

In the embodiment shown in FIGS. 7-9, the anchors 214, 215 are fixed into place on the support members 202 by tightening a set screw 218 against the support members 202. As described above, the support member 202 may include knurling or other textured surface. In these embodiments, an end of the set screw 218 may comprise a mating structure (e.g., teeth, protrusions, or the like) adapted to mate with knurling on the or otherwise enhance the fixation of the anchors 214, 215 to the support members 202.

A pair of spinous process connectors 208 is coupled to each offset rod 212 of the offset connectors 206. In some embodiments, at least one of the pair of spinous process connectors 208 is slidably coupled to the offset rod 212 and is moved axially along the offset rod 212 to secure the spinous process between the pair of spinous process connectors 208. In the embodiment shown in FIGS. 7-9, the spinous process connectors 208 each comprise a spinous process spiked plate 222 oriented facing each other. In this embodiment, each of the spinous process spiked plates 222 is movable axially with respect to each other along the offset rod to secure the superior spinous process between the pair of spinous process spiked plates 222. Each spinous process spiked plate 222 comprises fasteners 224 projecting from the spinous process spiked plate 222 toward the other spinous process spiked plate 222. The fasteners 224 engage the spinous process to fix the spinous process between the pair of spinous process spiked plates 222. The spinous process connectors 208 are fixed to the offset connectors 206 by a fastener, such as by tightening a set screw 226. As discussed above with respect to the support member 202, the offset rod 212 of the offset connector 206 may include textured (e.g., knurled) or smooth surface 210 for connection to the spinous process connectors 208. Similarly, the surface of the offset rods 212 may comprise any cross-section shape to assist in locking a spinous process connector 208 to the offset rod 212. In one embodiment, for example, the offset rod 212 may comprise a flat surface on which a set screw may be tightened.

The fasteners 224 may include sutures, wires, pins, straps, clamps, spikes, screws, teeth, adhesives, and/or other suitable fasteners. The fasteners may be integrated into the extensions or they may be modular. Modular fasteners may be adjustable, replaceable, and/or removable to allow tailoring of the kind and quality of fixation from rigid fixation to no fixation. The spinous process spiked plate and fasteners may advantageously be made of different materials. For example, the spinous process spiked plate may be made of a relatively softer material while the fasteners may be made of a relative harder material. For example, the spinous process spiked plate may be made of a polymer and/or other relatively soft material and the fastener may be made of a metal and/or other relatively hard material.

The fasteners 224 may take any suitable form. They may be made integral with the spinous process spiked plates 222, such as by machining or casting them with the plates 222, or they may be formed separately and permanently or removably attached to the spinous process spiked plates 222. In one embodiment, for example, fastener 224 is a sharpened spike that threadably engages the plate 222. The threaded engagement allows the fastener 224 to be replaced with a different fastener 224. For example, the fastener 224 may be replaced by one that has a different shape, a different size, a different material, or a different surface coating. The threaded engagement also allows the fastener 224 to be adjusted to extend by varying amounts from the plate 222 to vary how it engages the bone. Thus, the fastener 224 can be adjusted to fit differently shaped bones or to penetrate into a bone by varying amounts. For example, multiple threaded fasteners 224 can be adjusted to extend by different amounts to conform to curved or angled bone. Finally, the threaded engagement allows the user to remove the fastener 224 when fixation is not desired such as when it is desired to use implant 200 in a non-fusion procedure as an extension stop without limiting flexion.

Fasteners 224 can also be provided as multi-spike pods allowing a plurality of spikes to be quickly adjusted, changed, or omitted. Fastener 224 may include a non-circular tab engageable with a non-circular opening in the plate 222. In this embodiment, the non-circular engagement prevents the fastener 224 from rotating. The tab may form a press-fit, snap-fit, or other suitable engagement with the opening. The tab may be further secured by a supplemental screw. Fastener 224 includes a threaded shaft threadably engaged with a base member to allow the length of the fastener 224 to be adjusted. The shaft engages the plate 222 in rotating and pivoting manner such that the fastener 224 can be adjusted rotationally and angularly to engage the bone surface. In one embodiment, the shaft terminates in a spherical ball that engages the opening in a ball-and-socket arrangement for three degrees of freedom. However, any mechanism that allows any number of degrees of freedom may be used. The fastener 224 may be allowed to move in use so that as the plate 222 is pressed toward a bone the fastener 224 adjusts to the angle of the bone surface. The fastener 224 may also be secured such as by screw to adjust the tension in the joint and/or to lock the fastener 224 in a predetermined orientation.

In one embodiment, the pair of spinous process connectors 208 is coupled to the offset connector 206 via a ball socket 230 allowing freedom of movement to angle and/or rotate the spinous process spiked plates 222 with respect to the offset connector 206. The freedom of movement provided by the ball socket connection between the spinous process connectors 208 and the offset connector 206 allow the spinous process spiked plates 222 to be positioned to conform to curved or angled bone of the spinous process. In one embodiment, for example, the spinous process spiked plates 222 are able to be angled at least about ±20 degrees with respect to the offset connector 206. In a particular embodiment, the spinous process plates 22 are adapted to be angled at least about ±20 degrees in any direction with respect to offset connector 206 to provide a polyaxial cone of angulation. In an alternative embodiment, the spinous process plates 22 are adapted to be angled less than about ±20 degrees in any direction with respect to offset connector 206 to provide a polyaxial cone of angulation. Other connections allowing similar freedom of movement for the spinous process spiked plates 222 to be angled and/or rotated with respect to the offset connector 206 could also be provided. For example, the joint in the connection between the offset connector 206 and the spinous process spiked plates 222 may include enough free space through which the spinous process spiked plates may be angled and/or rotated with respect to the offset connector 206.

The segmental spinous process implants 100, 200 provide a flexible implant system that may be implanted in a patient in many configurations. The ability to longitudinally adjust the offset connector 106, 206 along the support member 102, 202 provides the ability to compress or distract disc space. In addition, the segmental spinous process implants 100, 200 provide for multilevel constructs with a single rigid construction to connect and secure multiple spinous processes. The spinous process implants 100, 200 further provide segmental spinal process anchors with modular connectors that allow fixation of a spinous process to one or more other spinous processes. Each spinal process anchor allows for independent fixation and manipulation of spinous processes (e.g., compression or distraction) and independent adjustment of the spinous process connectors at spinous processes of different vertebrae. While the Figures generally show spinous process connectors 108, 208 extending towards a superior spinous process, connectors 108, 208 could be oriented to extend towards an inferior spinous process. In some embodiments, spinous process connectors 108, 208 are adapted to receive fasteners 118, 218 in more than one orientation. This may be accomplished, for example, by having set screw receiving holes in two opposing sides of spinous process connectors 108, 208. Such an arrangement may allow a single spinous process connector 108, 208 to be coupled to either a superior or inferior spinous process.

Although embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A spinous process implant comprising:
a support member having a longitudinal axis;
an offset connector coupled to the support member, the offset connector comprising an anchor for selectively coupling the offset connector along the longitudinal axis of the support member and an offset member having a longitudinal axis extending at an angle away from the longitudinal axis of the support member, the offset member operable to extend laterally across a spine adjacent to at least one spinous process; and
a pair of opposing spinous process connectors operable to engage the spinous process, the pair of opposing spinous process connectors coupled to the offset member and extending away from the offset member operable to extend generally alongside either side of the spinous process,
wherein at least one of the pair of opposing spinous process connectors is movably coupled to the offset member so as to be movable with respect to the other opposing spinous process connector to secure the spinous process between the pair of opposing spinous process connectors.

2. The implant of claim 1 wherein the support member comprises a textured outer surface for engagement with the anchor of the offset connector.

3. The implant of claim 2 wherein the textured outer surface comprises a knurled outer surface.

4. The implant of claim 1 wherein the anchor and the offset member of the offset connector are integral.

5. The implant of claim 1 wherein the anchor is coupled to an outer surface of the support member.

6. The implant of claim 5 wherein the anchor is coupled to the outer surface of the support member via a set screw.

7. The implant of claim 1 wherein the longitudinal axis of the offset member is arranged generally transverse to the longitudinal axis of the support member.

8. The implant of claim 1 wherein the at least one of the pair of opposing spinous process connectors is slidably coupled to the offset member along the longitudinal axis of the offset member.

9. The implant of claim 8 wherein each of the pair of opposing spinous process connectors is slidably coupled to the offset member along the longitudinal axis of the offset member.

10. The implant of claim 1 wherein at least one of the pair of opposing spinous process connectors is coupled to the offset member via a ball socket.

11. The implant of claim 1 wherein the anchor of the offset connector comprises a closed connector slidably coupled to the support member and operable to be locked to the support member with a fastener.

12. The implant of claim 1 wherein the offset member comprises a tapered end opposite the anchor.

13. The implant of claim 1 wherein at least one of the pair of opposing spinous process connectors further comprises a fastener adapted to engage the spinous process.

14. The implant of claim 13 wherein the fastener comprises at least one spike adapted to engage the spinous process.

15. The implant of claim 1 wherein both of the pair of opposing spinous process connectors further comprise at least one fastener adapted to engage the spinous process.

16. The implant of claim 1 wherein the pair of opposing spinous process connectors are oriented to be coupled to a superior spinous process located superior to the offset member.

17. The implant of claim 1 wherein the pair of opposing spinous process connectors are oriented to be coupled to an inferior spinous process located inferior to the offset member.

18. The implant of claim 1 wherein at least one of the pair of opposing spinous process connectors is adapted to be angled between about zero degrees and about twenty degrees relative to the offset member.

19. The implant of claim 1 wherein at least one of the pair of opposing spinous process connectors is adapted to be angled more than about twenty degrees relative to the offset member longitudinal axis.

20. The implant of claim 1 wherein at least one of the pair of opposing spinous process connectors is adapted for polyaxial rotation relative to the offset member longitudinal axis.

21. The implant of claim 1 further comprising a second support member disposed on an opposite lateral side of the spinous process than the first support member, the second support member adapted to be coupled to the offset member.

22. The implant of claim 21 wherein the first and second support members are generally parallel when the offset connector is coupled to and between the first and second support members.

23. A method of using a spinous process implant, the method comprising:
providing a first elongate support member, an offset connector, and a pair of spinous process connectors;
slidably engaging the first elongate support member with the offset connector so that the offset connector is generally transverse to the elongate support member;
slidably engaging the pair of spinous process connectors with the offset connector, the pair of spinous process connectors extending generally transverse to the offset connector;
engaging a spinous process with the pair of spinous process connectors and fixing the position of the spinous process connectors to the offset connector to maintain the engagement with the spinous process; and
fixing the position of the offset connect to the first elongate support member.

24. The method of claim 23 wherein the slidably engaging of the pair of spinous process connectors with the offset connector further comprises adjusting an angle of at least one of the pair of spinous process connectors relative to a longitudinal axis of the offset connector.

25. The method of claim 23 wherein the offset connector further comprises an anchor disposed at a first end of the offset connector, and wherein the fixing of the position of the offset connector to the first elongate member comprises tightening a set screw disposed in the anchor to engage the first elongate member.

26. The method of claim 23 wherein the engaging of the spinous process comprises compressing at least one fastener disposed on at least one of the pair of spinous process connectors into the spinous process.

27. The method of claim 23 wherein the fixing of the position of the spinous process connectors to the offset connector comprises tightening a set screw disposed through the spinous process connector to engage the offset connector.

28. The method of claim 23 further comprising providing a second offset connector having a second pair of spinous process connectors.

29. The method of claim 28 further comprising coupling the second pair of spinous process connectors to a second spinous process.

30. The method of claim 29 further comprising distracting the first and second spinous processes from one another by translating at least one of the first and second offset connectors along the support member in a direction away from the other offset connector.

31. The method of claim 29 further comprising distracting the first and second spinous processes from one another by translating the first and second offset connectors along the support member in a direction away from each other.

32. The method of claim 29 further comprising compressing the first and second spinous processes towards one another by translating at least one of the first and second offset connectors along the support member in a direction towards the other offset connector.

33. The method of claim 29 further comprising compressing the first and second spinous processes towards one another by translating the first and second offset connectors along the support member in a direction towards each other.

34. The method of claim 23 further comprising providing a second elongate support member and engaging the second elongate member with the offset connector.

35. A bilateral spinous process implant comprising:
a first support member having a first longitudinal axis;
a second support member having a second longitudinal axis, the second support member spaced apart from the first support member;
an offset connector coupled to the first support member, the offset connector comprising (i) a first anchor for selectively coupling the offset connector along the first longitudinal axis of the first support member, (ii) a second anchor for selectively coupling the offset connector along the second longitudinal axis of the second support member and (iii) an offset member having a longitudinal axis extending between the first support member and the second support member, wherein the offset member is operable to extend laterally across a spine adjacent to at least one spinous process; and
a pair of opposing spinous process connectors operable to engage the spinous process, the pair of opposing spinous process connectors coupled to the offset member and extending away from the offset member to extend generally alongside either side of the spinous process,
wherein at least one of the pair of opposing spinous process connectors is movably coupled to the offset member so as to be movable with respect to the other opposing spinous process connector to secure the spinous process between the pair of opposing spinous process connectors.

36. The implant of claim 35 wherein the support member comprises a textured outer surface for engagement with the first anchor of the offset connector.

37. The implant of claim 35 wherein the offset connector comprises a textured outer surface for engagement with an anchor of at least one of the spinous process connectors.

38. The implant of claim 35 wherein at least one of the first and second anchors comprises a ball collet.

39. The implant of claim 35 wherein at least one of the pair of opposing spinous process connectors further comprises at least one spike adapted to engage the spinous process.

40. The implant of claim 35 wherein the first and second support members are generally parallel when the offset connector is coupled to the first and second support members.

41. The implant of claim 35 further comprising a second offset connector coupled to and extending between the first and second support members.
